(19) United States
(12) Patent Application Publication
(10) Pub. No.: US 2017/0097285 A1
(43) Pub. Date: Apr. 6, 2017

(54) SYSTEM AND METHOD FOR TESTING ULTRASOUND TRANSDUCER

(71) Applicant: Kona Medical, Inc., Bellevue, WA (US)

(72) Inventors: Joshua R. Doherty, Seattle, WA (US); David Nelson, Snohomish, WA (US); Michael Gertner, Menlo Park, CA (US); Tong Li, Seattle, WA (US); Jimin Zhang, Bellevue, WA (US)

(73) Assignee: Kona Medical, Inc., Bellevue, WA (US)

(21) Appl. No.: 14/876,805

(22) Filed: Oct. 6, 2015

(51) Int. Cl. G01M 99/00; H04N 5/225; H04N 5/33; G06T 7/00; A61N 7/02; G01J 5/02

(52) U.S. Cl. CPC G01M 99/002; A61N 7/02; G01J 5/02; H04N 5/33; G06T 7/0004; H04N 5/2252; A61N 2007/0052

(57) ABSTRACT

An apparatus for testing an ultrasound device having an ultrasound transducer and a controller includes: a housing; an absorbing layer inside the housing, wherein the absorbing layer is configured to receive ultrasound energy from the ultrasound transducer; and a thermal camera for detecting temperature at the absorbing layer. A method for testing an ultrasound device having an ultrasound transducer and a controller, includes: operating the ultrasound transducer to deliver ultrasound energy towards an absorbing layer at a testing apparatus using a thermal camera to detect temperature at the absorbing layer; obtaining thermal image data from the camera; and analyzing the thermal image data to determine whether the ultrasound device is operating desirably.

### FIELD

An embodiment described herein relates to system and method for testing an ultrasound device.

### BACKGROUND

An ultrasound device has been used to deliver ultrasound energy from outside a patient to treat different regions inside the patient. For example, ultrasound device has been used to deliver ultrasound energy to treat nerves (sympathetic nerves) around a blood vessel (e.g., renal artery) to treat hypertension. Such ultrasound device is unique in the sense that it is configured to deliver energy from an ultrasound transducer at a level that is sufficient to treat nerves while preserving the blood vessel surrounded by the nerves. Also, such ultrasound device is unique in that its operation requires a separate ultrasound imaging transducer to image the anatomy, to track the blood vessel, and to deliver energy at different regions around the blood vessel with certain precision.

A testing device and method for testing an ultrasound device that can easily determine the above power and targeting features would be of great value.

### SUMMARY

An apparatus for testing an ultrasound device containing an ultrasound transducer and a controller includes: a housing; an absorbing layer inside the housing, wherein the absorbing layer is configured to receive ultrasound energy from an ultrasound transducer; a thermal camera for detecting temperature at the absorbing layer; and an attenuating layer or layers positioned between the ultrasound device and the absorbing layer, wherein the attenuating layer(s) attenuate delivered ultrasound energy as to reduce the ultrasound energy incident at the absorbing layer.

Optionally, the ultrasound transducer is operated to deliver energy sequentially to a plurality of target areas at the absorbing layer.

Optionally, the target areas are arranged in a circular pattern, an elliptical pattern, a linear pattern, or any other defined pattern.

Optionally, the absorbing layer comprises a material with stable acoustic and mechanical properties in temperature environments ranging from 0 to 300° C.,

Optionally, the absorbing layer comprises a material with an acoustic velocity that is less than the velocity of the material proximal to the absorbing layer.

Optionally, the absorbing layer comprises a Teflon material.

Optionally, the absorbing layer comprises a product based of urethane, silicone, graphite, plastic, or any combination of the foregoing that may be mixed with various fillers possibly including polymeric or glass microspheres, boron-nitride, oxides, graphite, and/or the combination of any of the foregoing.

Optionally, the energy-attenuating device comprises one layer with zero thickness.

Optionally, the energy-attenuating device comprises two or more layers.

Optionally, the two or more layers comprise a first layer having a first thickness and a second layer having a second thickness, the first thickness being different from the second thickness and so on.

Optionally, the thickness of two or more layers are designed to attenuate the acoustic power in a way that the highest temperature in each layer is equal or close to be equal.

Optionally, the thickness of two or more layers are designed to attenuate the acoustic power in a way such that the amount of acoustic energy attenuated by each layer is approximately or close to equal.

Optionally, the energy-attenuating device comprises a silicone based product mixed with boron-nitride.

Optionally, the energy-attenuating device comprises a synthetic based product such as a plastic, urethane, or silicone material that may be mixed with various fillers possibly including polymeric or glass microspheres, boron-nitride, oxides, graphite, and/or the combination of any of the foregoing.

Optionally, the energy-attenuating device comprises a natural product such as wool felt or horse hair.

Optionally, the apparatus further includes one or more sensors attached to the energy-attenuating device.

Optionally, the one or more sensors are configured to sense one or more temperatures at the energy-attenuating device.

Optionally, the apparatus further includes a processing unit configured to obtain a first value from one of the one or more sensors, obtain a second value from the one of the one or more sensors, and determine a difference between the first value and the second value.

Optionally, the apparatus further includes a fiducial marker that is detectable using ultrasound imaging.

Optionally, the fiducial marker comprises a metal or plastic object attached to the absorbing layer.

Optionally, the apparatus further comprises a processing unit configured to determine a position of the fiducial marker, and to operate the ultrasound device based on the determined position.

Optionally, the apparatus further includes a processing unit configured to analyze thermal image data from the thermal camera to perform a power test for the ultrasound device.

Optionally, the processing unit is configured to calculate a first mean temperature for a first region-of-interest.

Optionally, the processing unit is configured to calculate a second mean temperature for a second region-of-interest.

Optionally, the processing unit is configured to determine a first set of data representing how temperature varies through time for a first region-of-interest.

Optionally, the processing unit is configured to determine a second set of data representing how temperature varies through time for a second region-of-interest.

Optionally, the processing unit is configured to determine a maximum temperature, a mean temperature, a slope of a temperature-vs-time curve, an integral value of temperatures through space, an integral value of temperatures through time, or any combination of two or more of the foregoing.

Optionally, the apparatus further includes a processing unit configured to analyze thermal image data from the thermal camera to perform a targeting test for the ultrasound device.

Optionally, the thermal image data is resulted from the ultrasound transducer delivering energy to multiple target areas in a defined pattern, and wherein the processing unit is configured to: determine locations of the respective target areas based on the thermal image data; calculate a mean location of the locations of the respective target areas; determine a difference between the mean location and a target location to obtain an overall targeting error for the defined pattern; and determine a difference between the mean location and the location of each respective target area to obtain a targeting error for each respective target area.

Optionally, the target areas are arranged in a circular pattern, an elliptical pattern, a linear pattern, or any other defined pattern.

Optionally, the apparatus further includes a processing unit configured to: obtain a first baseline temperature for a first region-of-interest before the ultrasound transducer is operated to deliver energy aiming at a first target area; obtain a first temperature data for the first region-of-interest after the ultrasound transducer is operated to deliver energy aiming at the first target area; and determine a first difference between the first temperature data and the first baseline temperature to obtain a first delta temperature.

Optionally, the processing unit is further configured to: obtain a second baseline temperature for a second region-of-interest before the ultrasound transducer is operated to deliver energy aiming at a second target area; obtain a second temperature data for the second region-of-interest after the ultrasound transducer is operated to deliver energy aiming at the second target area; and determine a second difference between the second temperature data and the second baseline temperature to obtain a second delta temperature.

Optionally, the apparatus further includes a processing unit configured to analyze thermal image data from the thermal camera to perform a power test and a targeting test for the ultrasound device

Optionally, the apparatus further includes a non-transitory medium for storing one or more images from the thermal camera.

Optionally, the apparatus further includes a non-transitory medium for storing temperature data obtained using the thermal camera.

Optionally, the apparatus further includes a processing unit for determining power performance of the ultrasound device based on results from a power test.

Optionally, the apparatus further includes a processing unit for determining targeting performance of the ultrasound device based on results from a targeting test.

Optionally, the apparatus further includes a processing unit for determining power performance and targeting performance of the ultrasound device based on results from a power test and targeting test.

Optionally, the apparatus further includes a camera holder for holding the thermal camera.

Optionally, the apparatus further includes a mounting component at or coupled to the housing for allowing the camera holder to be detachably secured thereto.

Optionally, the mounting component comprises a tubular structure defining a space for accommodating at least a part of the camera holder.

Optionally, the housing comprises a cover, and the mounting component is located at the cover.

Optionally, the housing defines a space for holding fluid.

Optionally, the housing includes side walls defining a perimeter of the housing, and a lid for covering an end of the housing.

Optionally, the housing comprises a mounting bracket.

Optionally, the mounting bracket is configured to align with the ultrasound device and to secure the apparatus to the ultrasound device.

A method for testing the power and targeting accuracy of an ultrasound device containing an ultrasound transducer and a controller includes: operating the ultrasound transducer to deliver energy towards an absorbing layer at a testing apparatus; using an energy-attenuating device positioned between the ultrasound transducer and the absorbing layer to attenuate the ultrasound energy delivered by the ultrasound transducer as to reduce the ultrasound energy incident at the absorbing layer; using a thermal camera to detect temperature at the absorbing layer; obtaining thermal image data from the camera; and analyzing the thermal image data to determine whether the ultrasound device is operating desirably.

Optionally, the ultrasound transducer is operated to deliver energy sequentially to a plurality of target areas at the absorbing layer.

Optionally, the target areas are arranged in a circular pattern, an elliptical pattern, a linear pattern, or any other defined pattern.

Optionally, the absorbing layer comprises a material with stable acoustic and mechanical properties in temperature environments ranging from 0 to 300° C.,

Optionally, the absorbing layer comprises a material with an acoustic velocity that is less than the velocity of the material proximal to the absorbing layer.

Optionally, the absorbing layer comprises a Teflon material.

Optionally, the absorbing layer comprises a product based of urethane, silicone, graphite, plastic, or any combination of the foregoing that may be mixed with various fillers possibly including polymeric or glass microspheres, boron-nitride, oxides, graphite, and/or the combination of any of the foregoing.

Optionally, the energy-attenuating device comprises one layer with zero thickness.

Optionally, the energy-attenuating device comprises two or more layers.

Optionally, the two or more layers comprise a first layer having a first thickness and a second layer having a second thickness, the first thickness being different from the second thickness and so on.

Optionally, the thickness of two or more layers are designed to attenuate the acoustic power in a way that the highest temperature in each layer is equal or close to be equal.

Optionally, the thickness of two or more layers are designed to attenuate the acoustic power in a way such that the amount of acoustic energy attenuated by each layer is approximately or close to equal.

Optionally, the energy-attenuating device comprises a silicone based product mixed the boron-nitride.

Optionally, the energy-attenuating device comprises a synthetic based product such as a plastic, urethane, or silicone material that may be mixed with various fillers possibly including polymeric or glass microspheres, boron-nitride, oxides, graphite, and/or the combination of any of the foregoing.

Optionally, the energy-attenuating device comprises a natural product such as wool felt or horse hair.

Optionally, the act of analyzing is for performing a power test for the ultrasound device.

Optionally, the act of analyzing comprises calculating a first mean temperature for a first region-of-interest.

Optionally, the act of analyzing further comprises calculating a second mean temperature for a second region-of-interest.

Optionally, the act of analyzing is performed to determine a first set of data representing how temperature varies through time for a first region-of-interest.

Optionally, the act of analyzing is performed to determine a second set of data representing how temperature varies through time for a second region-of-interest.

Optionally, the act of analyzing comprises determining a maximum temperature, a mean temperature, a slope of a temperature-vs-time curve, an integral value of temperatures through space, an integral value of temperatures through time, or any combination of two or more of the foregoing.

Optionally, the act of analyzing is for performing a targeting test for the ultrasound device.

Optionally, the ultrasound transducer is operated to deliver energy to multiple target areas in a defined pattern, and wherein the act of analyzing comprises: determining locations of the respective target areas based on the thermal image data; calculating a mean location of the locations of respective target areas; and determining a difference between the mean location and a target location to obtain an overall targeting error for the defined pattern; and determining a difference between the mean location and the location of each respective target area to obtain a targeting error for each respective targeting area.

Optionally, the target areas are arranged in a circular pattern, an elliptical pattern, a linear pattern, or any other defined pattern.

Optionally, the ultrasound transducer is operated to deliver energy aiming at a first target area and a second target area at the absorbing material, and wherein the act of analyzing comprises: obtaining a first baseline temperature for a first region-of-interest before the ultrasound transducer is operated to deliver energy aiming at the first target area; obtaining a first temperature data for the first region-of-interest after the ultrasound device is operated to deliver energy aiming at the first target area; and determining a first difference between the first temperature data and the first baseline temperature to obtain a first delta temperature.

Optionally, the act of analyzing further comprises: obtaining a second baseline temperature for a second region-of-interest before the ultrasound transducer is operated to deliver energy aiming at the second target area; obtaining a second temperature data for the second region-of-interest after the ultrasound transducer is operated to deliver energy aiming at the second target area; and determining a second difference between the second temperature data and the second baseline temperature to obtain a second delta temperature.

Optionally, the thermal image data is analyzed to perform a power test and a targeting test for the ultrasound device.

Optionally, the method further includes providing a tank of fluid between the ultrasound device and the absorbing layer.

An apparatus for testing an ultrasound device having an ultrasound transducer and a controller, includes: a housing; an absorbing layer inside the housing, wherein the absorbing layer is configured to receive ultrasound energy from the ultrasound transducer; and a thermal camera for detecting temperature at the absorbing layer.

Optionally, the ultrasound transducer is configured to deliver the ultrasound energy sequentially to a plurality of target areas at the absorbing layer.

Optionally, the target areas are arranged in a circular pattern, an elliptical pattern, a linear pattern, or any other defined pattern.

Optionally, the absorbing layer comprises a material that can withstand temperature ranging from 0° C. to 300° C.

Optionally, the housing comprises a compartment containing fluid, and the absorbing layer comprises a material with an acoustic velocity that is less than an acoustic velocity of the fluid.

Optionally, the absorbing layer comprises a Teflon material.

Optionally, the absorbing layer comprises a material that includes urethane, silicone, graphite, plastic, or any combination of the foregoing.

Optionally, the material is mixed with one or more fillers selected from the group consisting of polymeric microspheres, glass microspheres, boron-nitride, oxides, and graphite.

Optionally, the apparatus further includes an energy-attenuating device positioned between the ultrasound transducer and the absorbing layer, the energy-attenuating device configured to attenuate ultrasound energy provided by the ultrasound transducer to reduce an amount of the ultrasound energy received by the absorbing layer

Optionally, the energy-attenuating device comprises two or more layers.

Optionally, the two or more layers comprise a first layer having a first thickness and a second layer having a second thickness, the first thickness being different from the second thickness.

Optionally, the energy-attenuating device comprises a silicone based product mixed with boron-nitride.

Optionally, the energy-attenuating device comprises a plastic, urethane, or silicone material that is mixed with one or more fillers selected from the group consisting of polymeric microspheres, glass microspheres, boron-nitride, oxide, and graphite.

Optionally, the energy-attenuating device comprises a natural product.

Optionally, the apparatus further includes a fiducial marker that is detectable using ultrasound imaging.

Optionally, the fiducial marker comprises a metal or plastic object attached to the absorbing layer or to a component located in the housing.

Optionally, the ultrasound device is configured to determine a position of the fiducial marker, and to operate the ultrasound transducer based on the determined position.

Optionally, the apparatus further includes a processing unit configured to analyze thermal image data from the thermal camera to perform a power test for the ultrasound device.

Optionally, the processing unit is configured to calculate a first mean temperature for a first region-of-interest.

Optionally, the processing unit is configured to calculate a second mean temperature for a second region-of-interest.

Optionally, the processing unit is configured to determine a first set of data representing how temperature varies through time for a first region-of-interest.

Optionally, the processing unit is configured to determine a second set of data representing how temperature varies through time for a second region-of-interest.

Optionally, the processing unit is configured to determine a maximum temperature, a mean temperature, a slope of a temperature-vs-time curve, an integral value of temperatures through space, an integral value of temperatures through time, or any combination of two or more of the foregoing.

Optionally, the apparatus further includes a processing unit configured to analyze thermal image data from the thermal camera to perform a targeting test for the ultrasound device.

Optionally, the thermal image data is resulted from the ultrasound transducer delivering the ultrasound energy to multiple target areas in a defined pattern, and wherein the processing unit is configured to: determine locations of the respective target areas based on the thermal image data; calculate a mean location of the locations of the respective target areas; and determine a difference between the mean location and a target location to obtain an overall targeting error for the defined pattern.

Optionally, the processing unit is also configured to: determine a difference between the mean location and the location of at least one of the target areas to obtain a targeting error for the at least one of the target areas.

Optionally, the target areas are arranged in a circular pattern, an elliptical pattern, a linear pattern, or any other defined pattern.

Optionally, the apparatus further includes a processing unit configured to: obtain a first baseline temperature for a first region-of-interest before the ultrasound transducer is operated to deliver energy aiming at a first target area; obtain a first temperature data for the first region-of-interest after the ultrasound transducer is operated to deliver energy aiming at the first target area; and determine a first difference between the first temperature data and the first baseline temperature to obtain a first delta temperature.

Optionally, the processing unit is further configured to: obtain a second baseline temperature for a second region-of-interest before the ultrasound transducer is operated to deliver energy aiming at a second target area; obtain a second temperature data for the second region-of-interest after the ultrasound transducer is operated to deliver energy aiming at the second target area; and determine a second difference between the second temperature data and the second baseline temperature to obtain a second delta temperature.

Optionally, the apparatus further includes a processing unit configured to analyze thermal image data from the thermal camera to perform a power test and a targeting test for the ultrasound device.

Optionally, the apparatus further includes a non-transitory medium for storing one or more images from the thermal camera.

Optionally, the apparatus further includes a non-transitory medium for storing temperature data obtained using the thermal camera.

Optionally, the apparatus further includes a processing unit for determining power performance of the ultrasound device based on output from the thermal camera.

Optionally, the apparatus further includes a processing unit for determining targeting performance of the ultrasound device based on output from the thermal camera.

Optionally, the apparatus further includes a processing unit for determining power performance and targeting performance of the ultrasound device based on output from the thermal camera.

Optionally, the apparatus further includes a camera holder for holding the thermal camera.

Optionally, the apparatus further includes a mounting component at or coupled to the housing for allowing the camera holder to be detachably secured thereto.

Optionally, the mounting component comprises a tubular structure defining a space for accommodating at least a part of the camera holder.

Optionally, the housing comprises a cover, and the mounting component is located at the cover.

Optionally, the housing defines a space for holding fluid.

Optionally, the housing includes side walls defining a perimeter of the housing, and a lid for covering an end of the housing.

Optionally, the housing comprises a mounting bracket.

Optionally, the mounting bracket is configured to align with the ultrasound transducer and to secure the apparatus to the ultrasound transducer.

A method for testing an ultrasound device having an ultrasound transducer and a controller, includes: operating the ultrasound transducer to deliver ultrasound energy towards an absorbing layer at a testing apparatus; using a thermal camera to detect temperature at the absorbing layer; obtaining thermal image data from the camera; and analyzing the thermal image data to determine whether the ultrasound device is operating desirably.

Optionally, the method further includes using an energy-attenuating device positioned between the ultrasound transducer and the absorbing layer to attenuate the ultrasound energy delivered by the ultrasound transducer to reduce the ultrasound energy incident at the absorbing layer.

Optionally, the energy-attenuating device comprises two or more layers.

Optionally, the two or more layers comprise a first layer having a first thickness and a second layer having a second thickness, the first thickness being different from the second thickness.

Optionally, the energy-attenuating device comprises at least four layers.

Optionally, the energy-attenuating device comprises a silicone based product mixed with boron-nitride.

Optionally, the energy-attenuating device comprises a plastic, urethane, or silicone material that is mixed with one or more fillers selected from the group consisting of polymeric microspheres, glass microspheres, boron-nitride, oxide, and graphite.

Optionally, the energy-attenuating device comprises a natural product.

Optionally, the ultrasound transducer is operated to deliver the ultrasound energy sequentially to a plurality of target areas at the absorbing layer.

Optionally, the target areas are arranged in a circular pattern, an elliptical pattern, a linear pattern, or any other defined pattern.

Optionally, the absorbing layer comprises a material that can withstand temperature ranging from 0° C. to 300° C.

Optionally, the method further includes providing a container of fluid for coupling the ultrasound energy to the absorbing layer, wherein the absorbing layer comprises a material with an acoustic velocity that is less than the velocity of the fluid.

Optionally, the absorbing layer comprises a Teflon material.

Optionally, the absorbing layer comprises a material that includes urethane, silicone, graphite, plastic, or any combination of the foregoing.

Optionally, the material of the absorbing layer is mixed with one or more fillers selected from the group consisting of polymeric microspheres, glass microspheres, boron-nitride, oxide, and graphite.

Optionally, the act of analyzing is for performing a power test for the ultrasound device.

Optionally, the act of analyzing comprises calculating a first mean temperature for a first region-of-interest.

Optionally, the act of analyzing further comprises calculating a second mean temperature for a second region-of-interest.

Optionally, the act of analyzing is performed to determine a first set of data representing how temperature varies through time for a first region-of-interest.

Optionally, the act of analyzing is performed to determine a second set of data representing how temperature varies through time for a second region-of-interest.

Optionally, the act of analyzing comprises determining a maximum temperature, a mean temperature, a slope of a temperature-vs-time curve, an integral value of temperatures through space, an integral value of temperatures through time, or any combination of two or more of the foregoing.

Optionally, the act of analyzing is for performing a targeting test for the ultrasound device.

Optionally, the ultrasound transducer is operated to deliver the ultrasound energy to multiple target areas in a defined pattern, and wherein the act of analyzing comprises: determining locations of the respective target areas based on the thermal image data; calculating a mean location of the locations of the respective target areas; and determining a difference between the mean location and a target location to obtain an overall targeting error for the defined pattern.

Optionally, the method further includes determining a difference between the mean location and the location of at least one of the target areas to obtain a targeting error for the at least one of the target areas.

Optionally, the target areas are arranged in a circular pattern, an elliptical pattern, a linear pattern, or any other defined pattern.

Optionally, the ultrasound transducer is operated to deliver energy aiming at a first target area and a second target area at the absorbing material, and wherein the act of analyzing comprises: obtaining a first baseline temperature for a first region-of-interest before the ultrasound transducer is operated to deliver energy aiming at the first target area; obtaining a first temperature data for the first region-of-interest after the ultrasound transducer is operated to deliver energy aiming at the first target area; and determining a first difference between the first temperature data and the first baseline temperature to obtain a first delta temperature.

Optionally, the act of analyzing further comprises: obtaining a second baseline temperature for a second region-of-interest before the ultrasound transducer is operated to deliver energy aiming at the second target area; obtaining a second temperature data for the second region-of-interest after the ultrasound transducer is operated to deliver energy aiming at the second target area; and determining a second difference between the second temperature data and the second baseline temperature to obtain a second delta temperature.

Optionally, the thermal image data is analyzed to perform a power test and a targeting test for the ultrasound device.

Optionally, the method further includes providing a tank of fluid between the ultrasound transducer and the absorbing layer.

Other and further aspects and features will be evident from reading the following detailed description.

### BRIEF DESCRIPTION OF THE DRAWINGS

The drawings illustrate the design and utility of various features described herein, in which similar elements are referred to by common reference numerals. These drawings are not necessarily drawn to scale. In order to better appreciate how the above-recited and other advantages and objects are obtained, a more particular description will be rendered, which are illustrated in the accompanying drawings. These drawings depict only exemplary features and are not therefore to be considered limiting in the scope of the claims.

FIG. 1 illustrates a testing apparatus for testing an ultrasound device.

FIG. 2 illustrates a side view of the testing apparatus of FIG. 1.

FIG. 3 illustrates another side view of the testing apparatus of FIG. 1.

FIG. 4 illustrates the testing apparatus of FIG. 1.

FIG. 5 illustrates components of the testing apparatus of FIG. 1.

FIG. 6 illustrates a method for testing an ultrasound device.

FIG. 7 illustrates a method for performing a power test for an ultrasound device.

FIG. 8 illustrates a method for performing a targeting test for an ultrasound device.

FIG. 9 illustrates an example of a user interface that presents results of a power test and a targeting test.

FIG. 10 illustrates an example of temperature sensors attached to an energy attenuating device.

FIG. 11 illustrates a processing system with which embodiments described herein may be implemented.

### DETAILED DESCRIPTION

Various features are described hereinafter with reference to the figures. It should be noted that the figures are not drawn to scale and that the elements of similar structures or functions are represented by like reference numerals throughout the figures. It should be noted that the figures are only intended to facilitate the description of the features. They are not intended as an exhaustive description of the claimed invention or as a limitation on the scope of the claimed invention. In addition, an illustrated feature needs not have all the aspects or advantages shown. An aspect or an advantage described in conjunction with a particular feature is not necessarily limited to that feature and can be practiced in any other features even if not so illustrated.

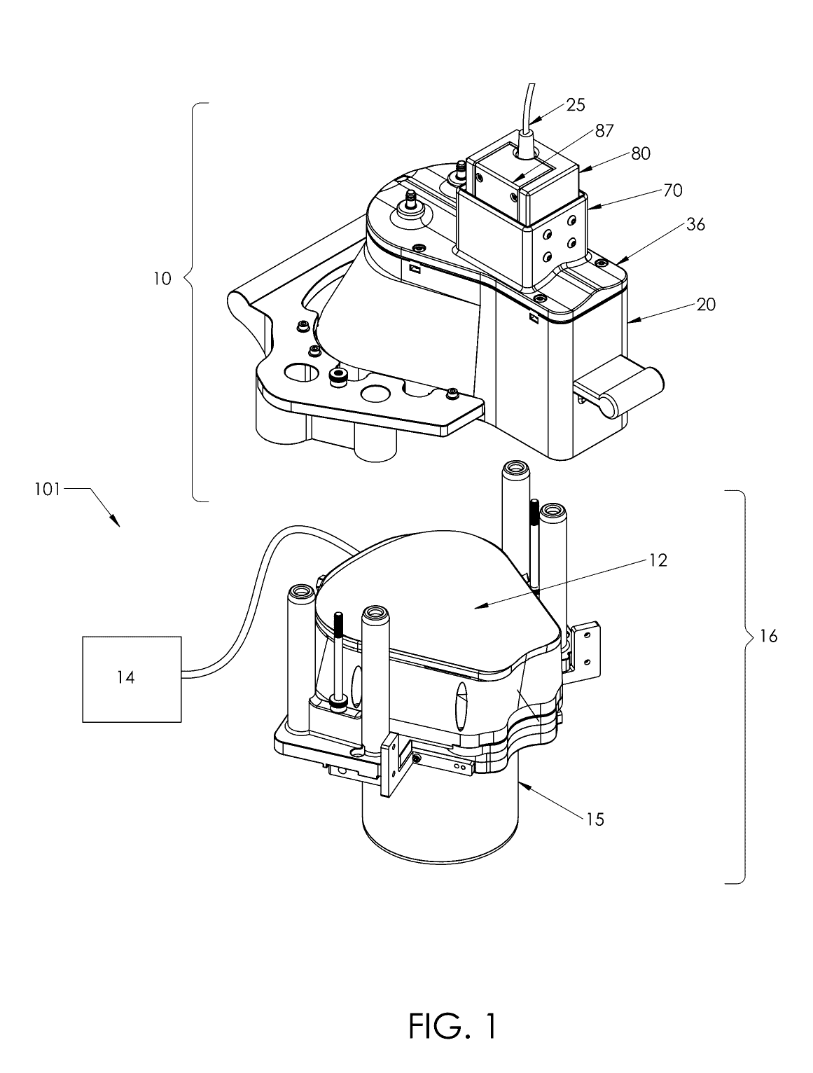

FIGS. 1-5 illustrate a testing apparatus 10 for testing an ultrasound device 101 containing an ultrasound transducer 12 and a controller 14 in accordance with some embodiments. In particular, FIG. 1 shows the testing apparatus 10 for coupling to the ultrasound transducer 12 so that the testing apparatus 10 can test different aspects of the ultrasound device 101. The ultrasound transducer 12 is mounted to a support 15, wherein the ultrasound transducer 12 and the support 15 together constitute a treatment module 16. In some embodiments, the ultrasound transducer 12 is a medical ultrasound transducer configured to deliver ultrasound energy from outside a patient to a region within the patient. Also, in some embodiments, the ultrasound transducer 12 is configured to deliver ultrasound energy to treat nerves around a blood vessel (e.g., a renal artery). Thus, in some embodiments, the ultrasound transducer 12 is configured to track a blood vessel (e.g., based on ultrasound imaging, or any of other types of imaging), and to deliver energy with sufficient precision to reach different regions around the blood vessel. In other embodiments, the ultrasound transducer 12 may be configured to deliver energy to treat other parts of the patient.

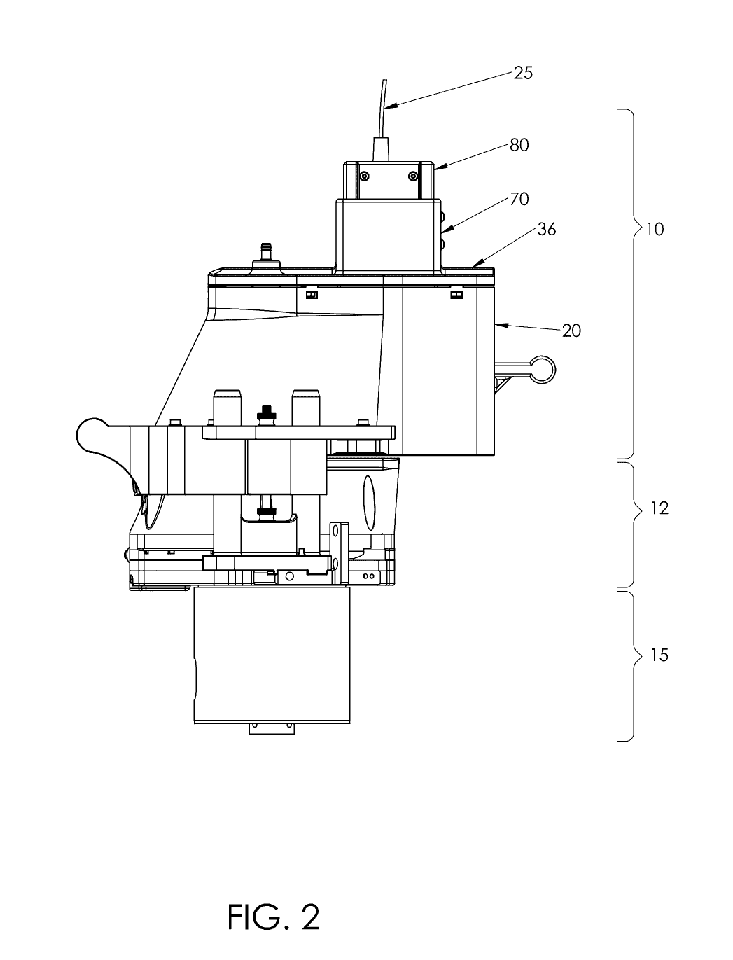

FIG. 2 illustrates a side view of the testing apparatus 10, particularly showing the testing apparatus 10 detachably coupled to the ultrasound transducer 12.

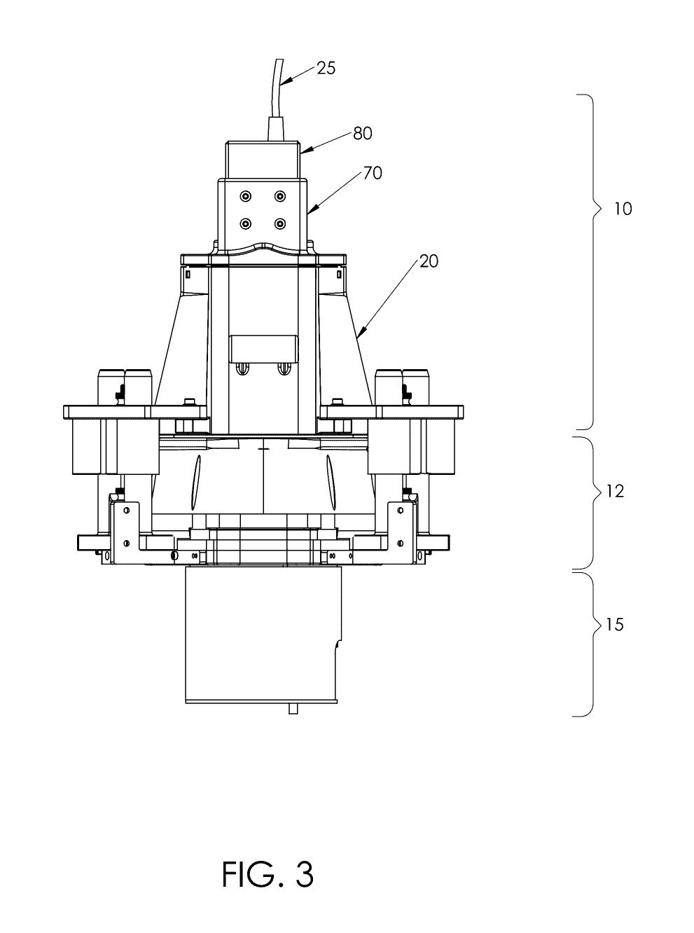

FIG. 3 illustrates another side view of the testing apparatus 10, particularly showing the testing apparatus 10 detachably coupled to the ultrasound transducer 12.

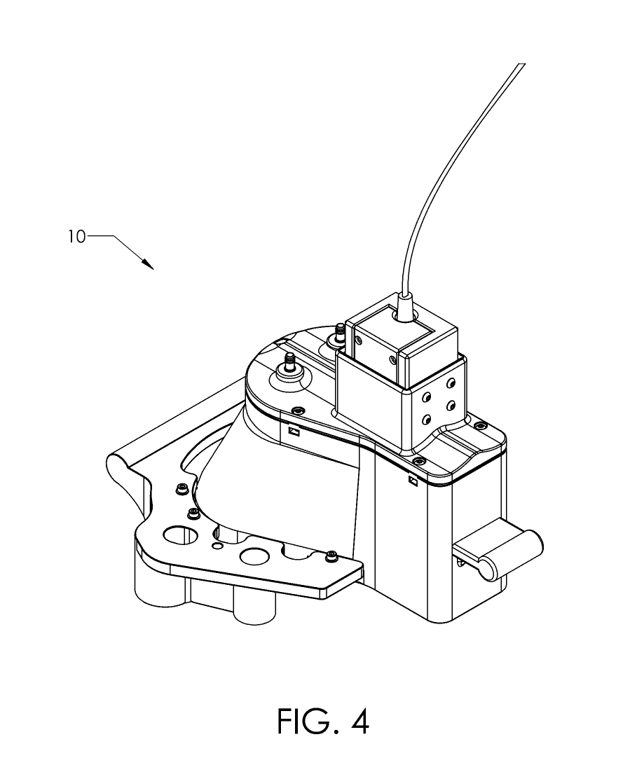

FIG. 4 shows the testing apparatus 10 by itself.

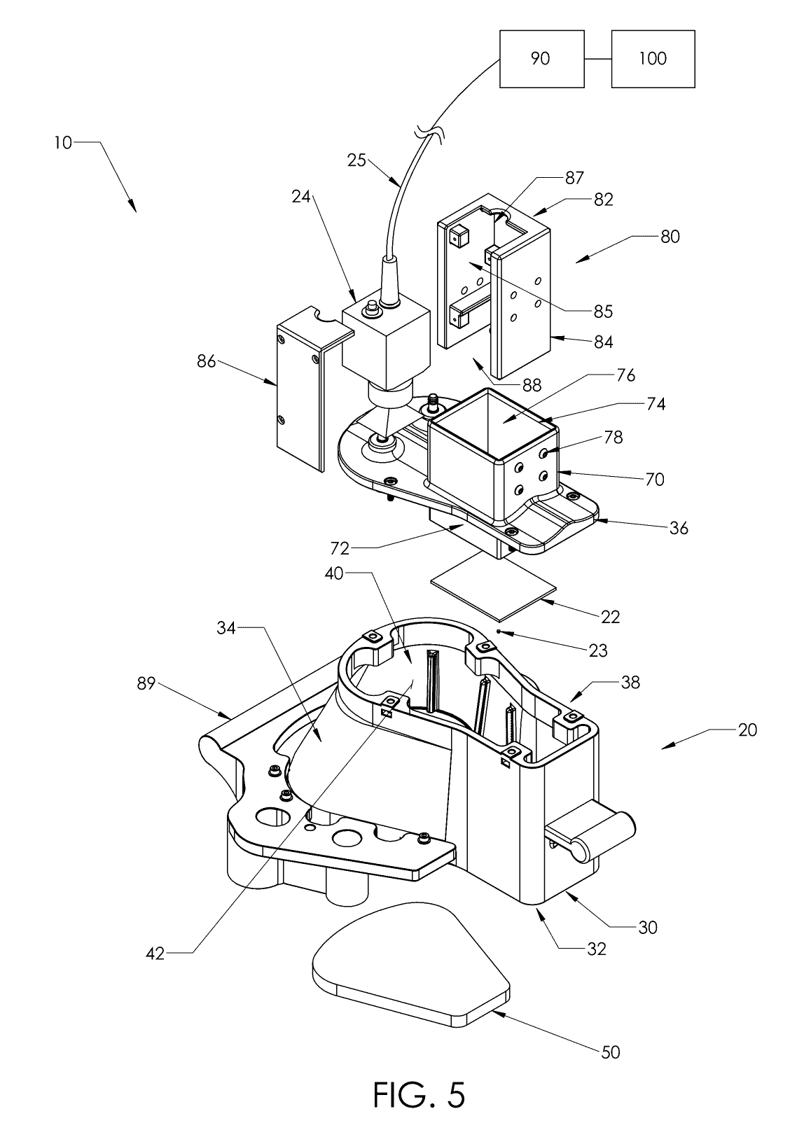

FIG. 5 illustrates components of the testing apparatus 10.

As shown in FIGS. 1-5, the apparatus 10 includes a housing 20, an absorbing layer 22 (shown in FIG. 5) configured to receive ultrasound energy from the ultrasound transducer 12, an energy-attenuating device 50 (shown in FIG. 5) configured to attenuate ultrasound energy delivered by the ultrasound transducer 12 as to reduce the ultrasound energy incident at the absorbing layer 22, and a thermal camera 24 for detecting temperature at the absorbing layer 22. It should be noted that as used in this specification, the term “layer” is not limited to a device having a single layer, and that the term “layer” may refer to any device or component having one or multiple layers. For example, the absorbing layer 22 may be a single layer in some embodiments, but may also include multiple sub-layers in other embodiments.

As shown in FIG. 5, the housing 20 includes a base 30 at a first end 32, side walls 34 defining a perimeter of the housing 20, and a cover (lid) 36 for covering the housing 20 at a second end 38. In other embodiments, the base 30 may be considered to be a separate component that is not a part of the housing 20. As shown in the figure, the base 30 and the side walls 34 together define a space 40 for containing fluid 42. The fluid 42 may be water (e.g., degassed water), saline, or any of other types of fluid. The fluid 42 in the housing 20 provides acoustic coupling to transmit ultrasound energy emitted from the ultrasound transducer 12 to the absorbing layer 22.

The absorbing layer 22 is configured to receive energy delivered from the ultrasound transducer 12. In some embodiments, the absorbing layer 22 comprises a Teflon material available at McMaster Carr. In other embodiments, the absorbing layer 22 may comprise a base product of urethane, silicone, graphite, plastic, or any combination of the foregoing. Also, in some embodiments, any of the foregoing materials, or combination of materials may be mixed with various fillers, such as those including polymeric and/or glass microspheres, boron-nitride, oxides, graphite, and/or the combination of any of the foregoing. The absorbing layer 22 is configured to absorb energy delivered from the ultrasound transducer 12. In some cases, the absorbing layer 22 should be able to withstand high temperatures, ranging from 0 to 300° C., and possess high thermal conductivity to allow the absorbing layer 22 to cool off quickly (e.g. within 10 seconds) after energy delivery stops.

In some embodiments, the absorbing layer 22 may have a thickness that is anywhere from 0.2 mm to 1.5 mm, and more preferably anywhere from 0.5 to 1.0 mm, and even more preferably anywhere from 0.6 to 0.9 mm, such as 0.8 mm. In other embodiments, the absorbing layer 22 may have a thickness that is more than 1.5 mm or less than 0.2 mm.

The absorbing layer 22 may be mechanically secured within the interior of the housing 20 (tank) while the absorbing layer 22 is suspended in the fluid 42 within the housing 20. As shown in FIG. 5, the testing apparatus 10 may include a mounting structure (mounting component) 70 having a first end 72 to which the absorbing layer 22 is coupled. In some embodiments, the absorbing layer 22 may be detachably coupled to the first end 72 of the mounting structure 70, e.g., through a clip, screw(s), etc. In other embodiments, the absorbing layer 22 may be permanently secured to the mounting structure 70 via an adhesive. The mounting structure 70 has a second end 74 for allowing the thermal camera 24 to be coupled thereto.

In the illustrated embodiments, the testing apparatus 10 also includes an energy-attenuating device 50 coupled to the housing 20. For example, the energy-attenuating device 50 may be attached to the interior surface, or the exterior surface, of the housing 20. The energy-attenuating device 50 is configured to attenuate energy output from the ultrasound transducer 12 so that the energy reaching the absorbing layer 22 is reduced. In other embodiments, the energy-attenuating device 50 may be formed as a part of the base 30. In further embodiments, the energy attenuating device 50 itself may be considered to be the base 30. Thus, the energy-attenuating device 50 may be a part of the housing 20, or a separate device that is coupled to the housing 20. In the illustrated embodiments, the energy-attenuating device 50 is a planar structure (e.g., a plate). In other embodiments, the energy-attenuating device 50 may have other configurations.

In some embodiments, the energy-attenuating device 50 comprises a synthetic based product such as plastic, urethane, or silicone that may be mixed with some partial materials or fillers possibly including polymeric or glass microspheres, boron-nitride, oxides, graphite, and/or the combination of any of the foregoing as to possibly increase the thermal conductivity or optimize the acoustic impedance of the energy-attenuating device 50. In other embodiments, the energy-attenuating device 50 comprises a natural product such as wool felt or horse hair. In other embodiments, the energy-attenuating device 50 may include any of other materials that is capable of attenuating at least some of the energy delivered from the ultrasound transducer 12.

Also, in the illustrated embodiments, the energy-attenuating device 50 comprises two or more layers. For example, the energy-attenuating device 50 may include a first layer having a first thickness and a second layer having a second thickness, the first thickness being different from the second thickness. In other example, the first thickness and second thickness may be the same. In other embodiments, the energy-attenuating device 50 may include only a single layer of material, or more than two layers of materials. In one implementation, the energy-attenuating device 50 includes four layers of materials. The four layers of materials at the energy-attenuating device 50 may be directly secured to each other, or may be separated from each other, e.g. by fluid or structural layers located between the energy-attenuating layers of the energy-attenuating device 50. Also, in some embodiments, each of the energy-attenuating layers at the energy-attenuating device 50 is configured to attenuate a same amount of energy (e.g., energy E±0.1 E). In other embodiments, each of the energy-attenuating layers at the energy-attenuating device 50 is configured to attenuate a different amount of energy.

In one implementation, the energy-attenuating layer device 50 may be a stack of 4 different thickness layers separated by a 5 mm distance. They are made of R-2949—a silicone based product mixed with boron-nitride for increased thermal conductivity, available at Nusil. The 4-layer stack and specific layer thicknesses are designed in a manner to attenuate specific amounts of energy in each layer. In some embodiments, the layer thicknesses range from 0.5 mm to 10 mm. For example, in some embodiments, the 4 layers have respective thicknesses of 1 mm, 1.5 mm 2.5 mm, and 6.0 mm for attenuating (absorbing) respective energies of 100 Watts, 90 Watts, 70 Watts, and 35 Watts, wherein the layer with the thickness of 1 mm is the closest to the ultrasound transducer, and the layer with the thickness of 6.0 mm is the furthest from the ultrasound transducer. In other embodiments, the layers may absorb different amount of energies. Also, in other embodiments, the energy-attenuating device 50 may include other silicone and urethane based materials mixed with varying amounts of graphite, glass or polymeric microspheres, oxides, boron-nitride and/or a combination of any of the foregoing and/or other fillers. In other embodiments, any of the layers in the energy-attenuating device 50 may have a thickness that is more than 10 mm or less than 0.5 mm. Also, in other embodiments, instead of having 4 layers, the energy-attenuating device 50 may have more than 4 layers or fewer than 4 layers (e.g., 3 layers, 2 layers, or 1 layer).

The combination of the energy-attenuating device 50 and the absorbing′ layer 22 is unique, as they depend on each other. For instance, if the energy-attenuating device 50 absorbs more energy, then the absorbing layer 22 does not need to withstand as high of temperatures, and vice versa. This is unique to the design—we need an energy-attenuating device 50 to attenuate enough energy, such that the energy reaching the absorbing layer 22 doesn't damage or modify the properties of that layer 22. In the illustrated embodiments, the testing apparatus 10 has the right combination of thickness and material properties in the energy-attenuating device 50 to deliver a reduced energy beam to the absorbing layer 22, and the right thickness and material property of the absorbing layer 22 that can withstand that power without being damaged or changing its properties.

In other embodiments, the energy-attenuating device 50 is not required, and the testing apparatus 10 may not include the energy-attenuating device 50.

Also, in the illustrated embodiments, the testing apparatus 10 further includes a fiducial marker 23 attached to the absorbing layer 22. In some embodiments the fiducial marker 23 may be metal or plastic. The fiducial marker 23 may have a spherical configuration, a semi-spherical configuration, a planar configuration, or any of other shapes. During use the fiducial marker 23 is detectable using ultrasound imaging or any other types of imaging. Ultrasound images may be analyzed by a processing unit to determine a position of the fiducial marker 23. Based on the determined position of the fiducial marker 23, the ultrasound transducer 12 is then operated to deliver energy to certain target areas with respect to the determined position of the fiducial marker 23. In some embodiments, the fiducial marker 23 may be secured to a surface of the absorbing layer 22. In other embodiments, the fiducial marker 23 may be embedded at least partially within a thickness of the absorbing layer 22. In further embodiments, the fiducial marker 23 may be secured to a separate planar device, which planar device is then coupled to the absorbing layer 22. In such cases, the fiducial marker 23 may be considered as being indirectly coupled to the absorbing layer 22. In other embodiments, the fiducial marker 23 may not be attached to the absorbing layer 22. Instead, the fiducial marker 23 may be attached to a component located in the housing. In such cases, the fiducial marker 23 may be proximal or distal to the absorbing layer 22 with respect to the ultrasound transducer.

It should be noted that the housing 20 of the testing apparatus 10 is not limited to the example shown in the figure, and that the housing 20 may have other configurations in other embodiments. For example, in other embodiments, the housing 20 may have a shape that is different from that shown.

As shown in FIG. 5, the testing apparatus 10 also includes a camera holder 80 configured to secure the thermal camera 24 relative to the housing 20 and/or the absorbing layer 22. In the illustrated embodiments, the camera holder 80 includes a camera housing 82. The camera housing 82 includes a first camera housing portion 84 defining a cavity 85 for accommodating the thermal camera 24, and a second camera housing portion 86. The first camera housing portion 84 also includes an opening 88 for allowing the thermal camera 24 to be placed there through and into the cavity 85. The second camera housing portion 86 is configured to couple to the first camera housing portion 84 to thereby cover the opening 88. In the illustrated example, the second camera housing portion 86 is in a form of a side wall. In other embodiments, the second camera housing portion may be a base of the camera housing 82, a cover of the camera housing 82, or any of other part(s) of the camera housing 82. As shown in the figure, the camera housing 82 also includes an opening 87 for allowing a cable 25 of the thermal camera 24 to extend therethrough. In some cases, the camera holder 80 may be considered to be a part of the thermal camera 24.

Also, as shown in FIG. 5, the mounting structure 70 at the cover 36 has an opening 76 at the second end 74 of the mounting structure 70 for allowing at least a part of the camera holder 80 to be inserted therethrough. After the camera holder 80 is placed in the mounting structure 70, fasteners 78 (e.g., screws) may then be used to secure the camera holder 80 relative to the cover 36 of the housing 20 through the mounting structure 70.

It should be noted that the camera holder 80 is not limited to the example shown in the figure, and that the camera holder 80 may have other configurations in other embodiments. For example, in other embodiments, the camera holder 80 may have a camera housing 82 having a different shape from that shown. Also, in other embodiments, the camera holder 80 may not include any camera housing 82. Instead, the camera holder 80 may include one or more structural members, such as one or more frames, one or more arms, one or more supports, etc., for securing the thermal camera 24 relative to the housing 20 and/or the absorbing layer 22.

Also, the mounting structure 70 is not limited to the example illustrated. In other embodiments, the mounting structure 70 may have other shapes and form. For example, in other embodiments, the mounting structure 70 may include a first component for securing the thermal camera 24/camera holder 80 relative to the housing 20, and a second component for holding the absorbing layer 22. The first and second components may be attached to each other, integrally formed together, or may be separate from each other (e.g., the mounting structure 70 may include a first mounting device for mounting the thermal camera 24/camera housing 80, and a second mounting device for mounting the absorbing layer 22).

As shown in FIG. 5, the housing 20 also includes a mounting bracket 89 configured to align with the ultrasound transducer 12 and to secure the apparatus to the ultrasound transducer 12.

As shown in the figure, the apparatus 10 further includes a non-transitory medium 90 for storing one or more images provided from the thermal camera 24. In one implementation, the medium 90 stores the spatial-temporal data from the thermal camera 24. The spatial-temporal data set constitutes a 3-D dataset (X, Y, and t) of temperature data through time. In the illustrated embodiments, the image(s) provided from the thermal camera 24 comprises thermal image(s), and the non-transitory medium 90 is for storing temperature data (temperature values for respective X, Y locations, and for certain time t) obtained using the thermal camera 24. The non-transitory medium 90 may be one or more storage devices located on a chip. Alternatively, the non-transitory medium 90 may be one or more external storage devices, such as one or more servers.

The testing apparatus 10 also includes a processing unit 100 configured to perform power test, targeting test, or both, for the ultrasound device 101 based on the thermal image data output from the thermal camera 24.

In some embodiments, the processing unit 100 may be the same processing unit, or may be implemented using the processing unit in the controller 14 that controls the operation of the ultrasound transducer 12. In other embodiments, the processing unit 100 may be a separate device that is different from the processing unit in the controller 14. Also, the processing unit may be implemented using one or more processors, such as a FPGA processor, an ASIC processor, a microprocessor, a signal processor, a general purpose processor, or any of other types of processor. In some cases, the processing unit may be considered an improved processing unit compared to known processing units because the processing unit described herein contains features, functions, and/or capabilities that are believed to be unavailable in known processing units.

In some cases, both the non-transitory medium 90 and the processing unit 100 may be integrated into a module, such as a hardware chip.

In some embodiments, the processing unit 100 is configured to determine the power performance based on an amount of temperature at the absorbing layer 22. In one implementation, the controller 14 (the processing unit in the controller 14) is configured to operate the ultrasound transducer 12 to deliver energy sequentially to a plurality of target areas at the absorbing layer 22. The processing unit in the controller 14 may prescribe a plurality of target areas to be aimed (through phasing and/or mechanical positioning) by the ultrasound transducer 12 at the absorbing layer 22. The prescribed target areas may be arranged in a circular pattern, an elliptical pattern, a linear pattern, or any of other patterns. During the energy delivery session, the thermal camera 24 monitors the temperature at each of the target areas at the absorbing layer 22, and generates thermal image data accordingly. The processing unit 100 is configured to analyze the thermal image data from the thermal camera 24 to perform a power test for the ultrasound device 101.

In some embodiments, the processing unit 100 is configured to calculate multiple mean temperatures for different respective regions-of-interest (ROIs) at the absorbing layer 22. For example, the processing unit 100 may calculate a first mean temperature for a first region-of-interest corresponding to a first target area aimed by the ultrasound transducer 12, and to calculate a second mean temperature for a second region-of-interest corresponding to a second target area aimed by the ultrasound transducer 12.

Also, in some embodiments, the processing unit 100 may be configured to determine multiple sets of data representing how temperature varies through time for different respective ROIs. For example, the processing unit 100 may be configured to determine a first set of data representing how temperature varies through time for a first region-of-interest corresponding to a first target area aimed by the ultrasound transducer 12, and to determine a second set of data representing how temperature varies through time for a second region-of-interest corresponding to a second target area aimed by the ultrasound transducer 12.

It should be noted that the processing unit 100 is not limited to determining mean temperatures for carrying out the power test for the ultrasound device 101, and that the processing unit 100 may be configured to determine other parameters for testing the power of the ultrasound device 101. For example, the processing unit 100 may be configured to determine a maximum temperature, a mean temperature, a slope of a temperature-vs-time curve, an integral value of temperatures through space, an integral value of temperatures through time, or any combination of two or more of the foregoing, for each of the ROIs at the absorbing layer 22 corresponding to the different respective target areas aimed by the ultrasound transducer 12.

In some embodiments, instead of or in addition to, performing power test for the ultrasound device 101, the processing unit 100 may also be configured to analyze thermal image data from the thermal camera 24 to perform a targeting test for the ultrasound device 101.

In one implementation, the controller 14 (the processing unit in the controller 14) is configured to operate the ultrasound transducer 12 to deliver energy sequentially to a plurality of target areas at the absorbing layer 22. The processing unit in the controller 14 may prescribe a plurality of target areas to be aimed (through phasing and/or mechanical positioning) by the ultrasound transducer 12 at the absorbing layer 22. The prescribed target areas may be arranged in a circular pattern, an elliptical pattern, a linear pattern, or any of other patterns. During the energy delivery session, the thermal camera 24 monitors the temperature at each of the target areas at the absorbing layer 22, and generates thermal image data accordingly. The processing unit 100 is configured to analyze the thermal image data from the thermal camera 24 to perform a targeting test for the ultrasound device 101.

In some embodiments, the thermal image data provided from the thermal camera 24 is resulted from the ultrasound transducer 12 delivering energy to multiple target areas (prescribed by a testing algorithm). In one implementation, the prescribed target areas are arranged in a circular pattern surrounding a prescribed center. In such cases, the thermal image data provided from the thermal camera 24 will indicate the locations of multiple targeted areas. If the ultrasound transducer 12 operates correctly, the multiple targeted areas will also be in a circular pattern. The processing unit 100 may be configured to determine locations of the respective targeted areas based on the thermal image data, and to calculate a mean location of the locations. If the ultrasound transducer 12 operates correctly, the mean location (X, Y) will provide a location of a center of the targeted areas that coincides with the prescribed center. The processing unit 100 may be configured to determine a difference between the mean location and a prescribed target location (prescribed center around which the prescribed targets surround) to obtain a targeting error. In some embodiments, the processing unit 100 may be configured to determine a difference between the mean location (X,Y) and the location of each of the multiple targeted areas to obtain a targeting error.

It should be noted that as the ultrasound transducer 12 sequentially delivers energy to the different prescribed target areas surrounding a prescribed center, the temperature spread from one targeted area may reach the location of another targeted area. Also before energy is delivered to a certain target area at the absorbing layer 22, that target area may already have a baseline temperature, which may be due to the heat generated at the testing apparatus 10, the temperature of the surrounding environment, and/or temperature resulted from energy delivery at other target area(s). In some cases, to consider the temperature due to only energy delivery, the baseline temperature before the energy delivery may be determined and later taken into account.

For example, the processing unit 100 may be configured to: obtain a first baseline temperature for a first region-of-interest before the ultrasound transducer 12 is operated to deliver energy aiming at a first target area, obtain a first temperature data for the first region-of-interest after the ultrasound transducer 12 is operated to deliver energy aiming at the first target area, and determine a first difference between the first temperature data and the first baseline temperature to obtain a first delta temperature. The first delta temperature may then be used by the processing unit 100 to determine parameters for the first region-of-interest for the power test and/or the targeting test for the ultrasound device 101. Also, the processing unit 100 may be configured to: obtain a second baseline temperature for a second region-of-interest before the ultrasound transducer 12 is operated to deliver energy aiming at a second target area; obtain a second temperature data for the second region-of-interest after the ultrasound transducer 12 is operated to deliver energy aiming at the second target area; and determine a second difference between the second temperature data and the second baseline temperature to obtain a second delta temperature. The second delta temperature may then be used by the processing unit 100 to determine parameters for the second region-of-interest for the power test and/or the targeting test for the ultrasound device 101.

Similarly, baseline temperatures at the different ROIs may be determined and taken into account when determining parameters for the power test and/or targeting test.

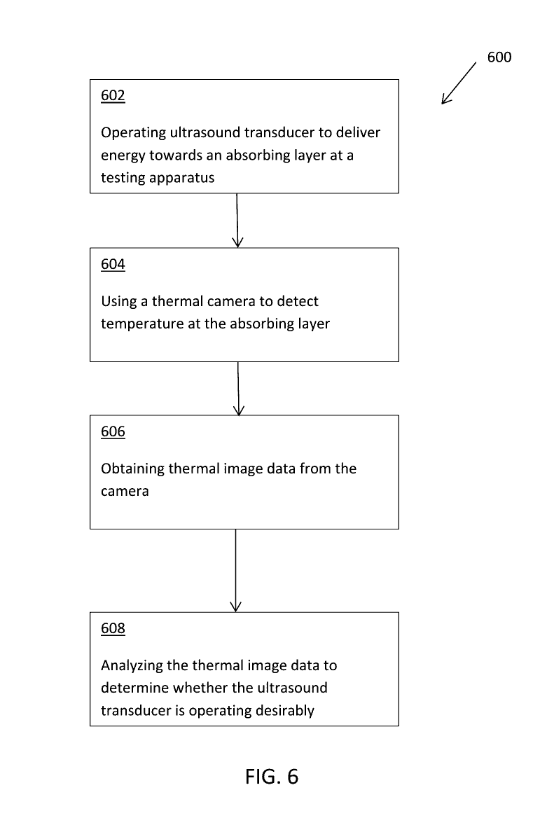

FIG. 6 illustrates a method 600 for testing an ultrasound device. The method 600 includes operating the ultrasound device 101 to deliver energy towards an absorbing layer 22 at a testing apparatus 10 (item 602), using a thermal camera 24 to detect temperature at the absorbing layer 22 (item 604), obtaining thermal image data from the thermal camera 24 (item 606), and analyzing the thermal image data to determine whether the ultrasound device 101 is operating desirably (item 608).

In some embodiments, in item 602, the ultrasound device 101 is operated to deliver energy sequentially to a plurality of target areas at the absorbing layer 22. In the illustrated embodiments, the controller 14 may operate the ultrasound transducer 12 to deliver energy to multiple target areas based on prescribed target areas arranged in a circular pattern. For example, a test algorithm in the controller 14 may prescribe a center and fourteen target areas surrounding the prescribed center at a certain radius R from the prescribed center. In such cases, the controller 14 then operates the ultrasound transducer 12 to deliver energy sequentially to fourteen areas at the absorbing layer 22 in accordance with the fourteen prescribed target areas, with the prescribed center being placed at the fiducial marker 23 at the absorbing layer 22. In particular, the controller 14 may use ultrasound imaging (or any other types of imaging) to identify the position of the fiducial marker 23 at the absorbing layer 22. Then, based on the test algorithm, the controller 14 then treats the position of the fiducial marker 23 as the prescribed center, and controls the ultrasound transducer 12 to deliver energy sequentially to fourteen areas located at radius R from the center. The prescribed radius R may be a value that is anywhere from 3 mm to 9 mm. In other examples, the radius R may be higher than 9 mm or less than 3 mm. In other embodiments, the prescribed target areas may be arranged in an elliptical pattern, a linear pattern, or any of other patterns. Also, in other embodiments, the number of prescribed target areas may be more or fewer than fourteen.

Also, in some embodiments, in item 606, the thermal image camera 24 provides thermal image data (one or more images showing temperature information) for each of the prescribed target areas. For example, before energy is delivered by the ultrasound device 101 to a first prescribed target area at the absorbing layer 22, the thermal camera 24 may provide a first thermal image of the absorbing layer 22 establishing a base line temperature for the first prescribed target area. The base line temperature represents the temperature of the region in the absorbing layer 22 corresponding to the first prescribed target area that is not attributable to the energy delivery by the ultrasound transducer 12. Then while energy is being delivered from the ultrasound transducer 12 to the first prescribed target area, the thermal camera 24 may provide multiple thermal images showing how the temperature in a first region-of-interest at the absorbing layer 22 varies over time as energy is being delivered to the first prescribed targeted area. The processing unit 100 may subtract the base line temperature established earlier from the temperature data in these thermal images to determine the actual temperature values in these thermal images that are attributable to the energy delivered from the ultrasound transducer 12. In some embodiments, the act of subtracting the base line temperature from the temperature data may be performed by the processing unit 100 in item 608 as a part of the act of analyzing the thermal image data. In one implementation, the processing unit 100 may include a base line temperature determination module configured to determine base line temperatures for the respective prescribed targeted areas.

In addition, in some embodiments, in item 608, the processing unit 100 may perform a power test, a targeting test, or both, for the ultrasound device 101.

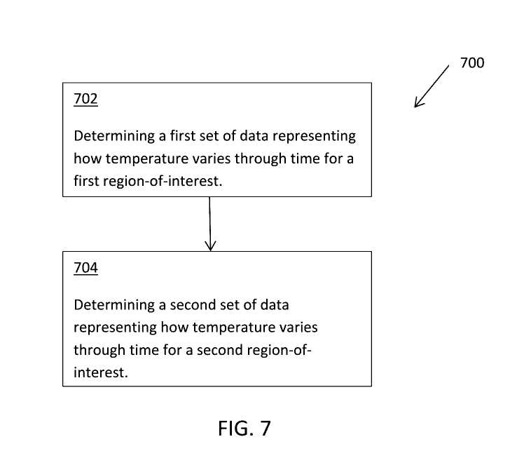

In accordance with some embodiments, to perform a power test for the ultrasound device 101, the processing unit 100 may include a power test module configured to calculate mean temperatures for different respective regions-of-interest (ROIs) corresponding to the different respective target areas. For example, as shown in FIG. 7, the act of analyzing in item 608 may be performed in a power test method 700 by the power test module in the processing unit 100, which calculates a first mean temperature for a first region-of-interest (item 702), and calculates a second mean temperature for a second region-of-interest (item 704). In some cases, the mean temperature for a certain region-of-interest (ROI) may be calculated after the base line temperature values for that ROI is subtracted from the temperature values in the ROI of the thermal image. For example, if the base line temperature values in the ROI (3×3 pixels) is:

323

122

121

Also, if the thermal image provided (output from the thermal camera 24 after energy is delivered) has the following values in the same ROI:

645

696

766

Then the adjusted temperature values in the ROI will be calculated by the power test module as:

$$\begin{matrix}645 \\ 696 \\ 766\end{matrix} - \begin{matrix}323 \\ 122 \\ 121\end{matrix} = \begin{matrix}322 \\ 574 \\ 645\end{matrix}$$

The adjusted temperature values in the ROI represent the change in temperature caused by the delivered energy from the ultrasound transducer 12. The mean temperature may then be calculated as (3+2+2+5+7+4+6+4+5)/9=4.2 for the ROI. The same process may be performed for other ROIs. For example, if a test algorithm prescribes fourteen target areas, then the processing unit 100 may determine fourteen corresponding ROIs, and the above process may be repeated fourteen times for the fourteen respective ROIs by the power test module in the processing unit 100.

Also, in some embodiments, to perform the power test for the ultrasound device 101, the power test module in the processing unit 100 may analyze the thermal image data to determine multiple sets of data representing how temperatures at different respective ROIs vary through time. For example, the power test module in the processing unit 100 may determine a first set of data representing how temperature varies through time for a first region-of-interest, and may determine a second set of data representing how temperature varies through time for a second region-of-interest. The temperature values in the temperature-versus-time data set may include thermal image data values, adjusted temperature values, mean temperatures, maximum temperature values, integral temperature values through space, integral temperature values through time, for the respective ROIs. Thus, in some embodiments, the power test module in the processing unit 100 may be configured to determine a maximum temperature, a mean temperature, a slope of a temperature-vs-time curve, an integral value of temperatures through space, an integral value of temperatures through time, or any combination of two or more of the foregoing, for each of the ROIs.

In some embodiments, the testing apparatus 10 may further include a display presenting a graphical user interface, which allows results from the power test to be presented to a user. For example, the user interface may present a plot, based on output from the power test module, showing how temperature varies through time for one of the ROIs, a plurality of ROIs, or all of the ROIs.

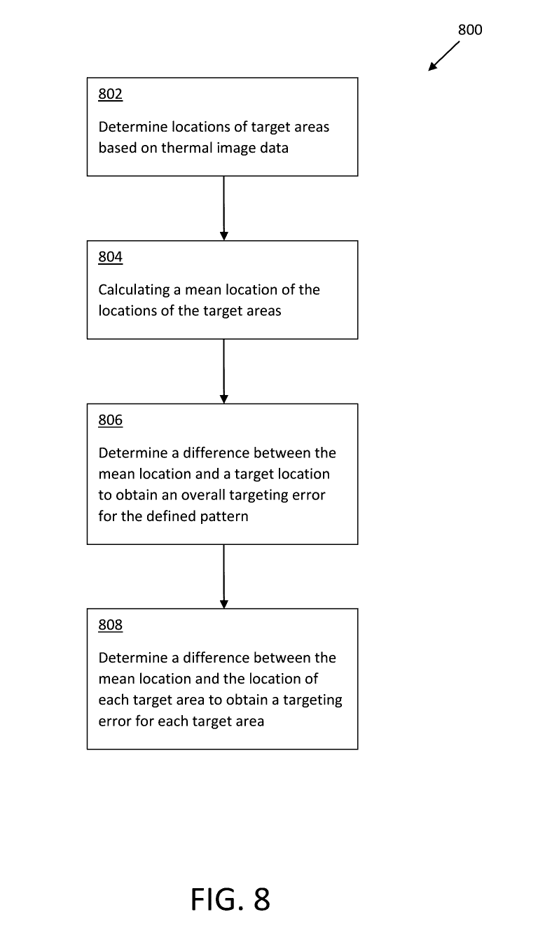

FIG. 8 illustrates a method 800 for performing a targeting test for an ultrasound device 101. The method 800 includes: determining locations of target areas based on thermal image data (item 802), calculating a mean location of the locations of the target areas (item 804), determining a difference between the mean location and a target location to obtain an overall targeting error for the defined pattern (item 806). In some cases, the target location in item 806 represents a prescribed location around which the multiple prescribed target positions are defined. In an actual treatment, the target location may be a location at the blood vessel (e.g., within a lumen, such as a center, of the blood vessel), and the prescribed target positions are around such target location. As shown in the figure, the method 800 also includes determining a difference between the mean location (e.g., the center of the pattern) and the locations of each target area to obtain a targeting error for each target area (item 808). In some cases, item 808 is optional, and the method 800 may not include item 808.

In one implementation, to perform a targeting test for the ultrasound device 101, the processing unit 100 may include a targeting test module, which in item 802, analyzes thermal images from the thermal camera 24 to determine positions of the respective target area (e.g., positions of the respective heat zones) at the absorbing layer 22. In the illustrated embodiments, the controller 14 is configured to control the ultrasound transducer 12 based on a test algorithm to deliver energy sequentially to multiple target areas on the absorbing layer 22 in some defined pattern. In one implementation, the prescribed target areas are arranged in a circular pattern surrounding a prescribed center. In such cases, the thermal image data provided from the thermal camera 24 will indicate the locations of multiple targeted areas. If the ultrasound device 101 operates correctly, the multiple targeted areas will also be in a circular pattern. The targeting test module in the processing unit 100 may be configured to determine locations of the respective targeted areas based on the thermal image data, and to calculate a mean location of the locations of the respective targeted areas in item 804. If the ultrasound device 101 operates correctly, the mean location (X, Y) will provide a location of a center of the targeted areas that coincides with the prescribed center. The targeting test module in the processing unit 100 may be configured to determine a difference between the mean location and a prescribed target location (prescribed center around which the prescribed targets surrounds) to obtain an overall targeting error for the defined pattern in item 806. The value of the difference may indicate a degree of targeting error. In some embodiments the targeting test module in the processing unit 100 may be configured to determine a difference between the mean location and the location of each target area to obtain a targeting error for each target area in item 808. The value of the difference may indicate a degree of targeting error.

In some embodiments, the testing apparatus 10 may further include a display presenting a graphical user interface, which allows results from the targeting test to be presented to a user. For example, the user interface may present a plot, based on output from the targeting test module, showing the locations, and/or temperature values, of multiple targeted areas at the absorbing layer 22.

In some embodiments, when both the power test and the targeting test are performed for the ultrasound device 101, the ultrasound transducer 12 may deliver the sequence of energy once to the plurality of target areas for both tests. Then thermal image data resulted from such energy delivery may be analyzed to obtain test results for both the power test and the targeting test. In other embodiments, the ultrasound transducer 12 may deliver the sequence of energy once to the plurality of target areas for the power test, and then may deliver the sequence of energy again to the plurality of target areas for the targeting test. In such embodiments, the plurality of target areas for the power test may be the same or different from the plurality of target areas for the targeting test.

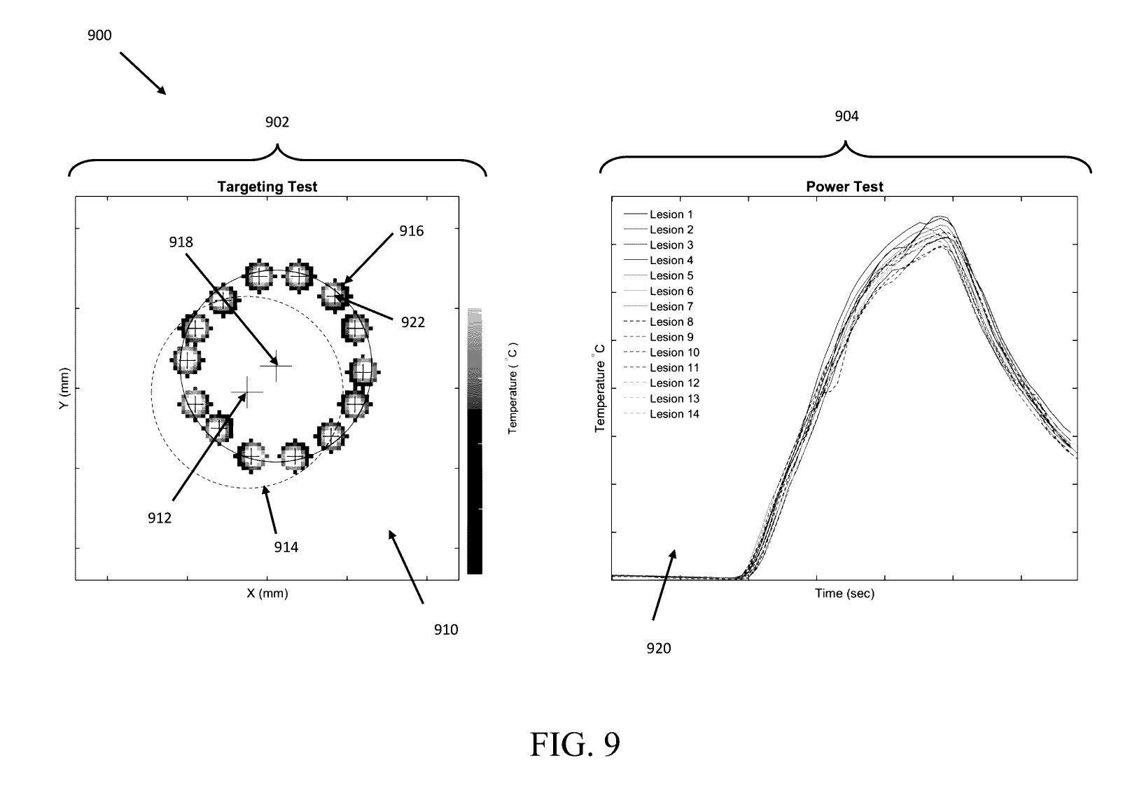

FIG. 9 illustrates an example of a user interface 900 that presents results of the power test and targeting test. The user interface 900 includes a first area 902 showing results from the targeting test, and a second area 904 showing results from the power test.

In the first area 902, an image 910 is presented showing a prescribed center 912, a prescribed circular pattern with a defined radius 914, actual positions of the heat zones 916 (target areas), actual center positions 922 of the respective heat zones 916, and actual center 918 of the pattern of the heat zones 916. As used in this specification, the term “center” is not necessarily limited to a point or location that is the middle of a circle, and may be used to refer to any point or location that is within any shape defined based on certain user-defined criteria. As discussed, a test algorithm in the controller 14 may prescribe a center 912 and fourteen target areas surrounding the prescribed center at a certain radius 914 from the prescribed center 912. In such cases, the controller 14 then operates the ultrasound transducer 12 to deliver energy sequentially to fourteen areas at the absorbing layer 22 in accordance with the fourteen prescribed target areas, with the prescribed center 912 being placed at the fiducial marker 23 at the absorbing layer 22. In particular, the controller 14 may use ultrasound imaging to identify the position of the fiducial marker 23 at the absorbing layer 22. Then, based on the test algorithm, the controller 14 then treats the position of the fiducial marker 23 as the prescribed center 912, and controls the ultrasound transducer 12 to deliver energy sequentially to fourteen areas located at radius 914 from the center. In other embodiments, the prescribed target areas may be arranged in an elliptical pattern, a linear pattern, or any of other patterns. Also, in other embodiments, the number of prescribed target areas may be more or fewer than fourteen.

As shown in FIG. 9, the thermal image data provided from the thermal camera 24 indicate the locations of multiple targeted areas 916. If the ultrasound device 101 operates correctly, the multiple targeted areas 916 will also be in a circular pattern. The processing unit 100 may be configured to determine locations of the respective targeted areas 916 based on the thermal image data, and to calculate a mean location of the locations in item 804. If the ultrasound device 101 operates correctly, the mean location (X, Y) will provide a location of a center 918 of the targeted areas that coincides with or is within some prescribed tolerance (e.g. <2 mm) from the prescribed center 912. The processing unit 100 may be configured to determine a difference between the mean location 918 and a prescribed target location (prescribed center 912 around which the prescribed targets surrounds) to obtain an overall targeting error for the pattern in item 806. The targeting error is show graphically as the offset between the prescribed center 912 and the actual center 918. In some embodiments the processing unit 100 may be configured to determine a difference between the mean location 918 and the center location of each target area 922 to obtain a targeting error in item 808.

In the second area 904 of the user interface 900, a plot 920 showing how temperature varies through time for the fourteen target areas is presented. In the illustrated example, the spatial-mean delta temperatures through time within a region-of-interest (ROI) surrounding each lesion at the absorbing layer 22 is presented in the plot 920. This represents the delta temperature through time plot for each lesion in the figure. It can be seen that the temperatures increase starting at some time (corresponding to the start of energy delivery) and continue to increase for some duration until the ultrasound transducer 12 is turned off and the temperatures quickly decrease. The temperature values in the temperature-versus-time data set are not limited to mean temperature values. In other embodiments, the temperature values in the temperature-versus-time data set may include thermal image data values, adjusted temperature values, maximum temperature values, integral temperature values through space, integral temperature values through time, for the respective ROIs. As shown in the figure, the plot 920 includes multiple curves for the respective target areas. If the ultrasound device operates correctly, the measured temperatures should fall within some prescribed tolerance (e.g. +/−3° C.) of a calibrated or anticipated value. Assuming the same amount of power was delivered to each target area, then the temperature-versus time plot 920 for each target area should be approximately similar in slope and peak temperature value reached if the ultrasound device is operating correctly.

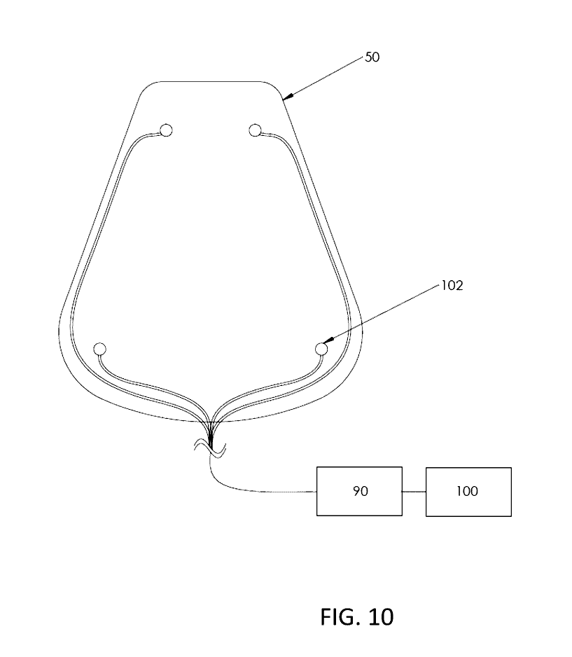

In any of the embodiments described herein, the energy attenuating device 50 may optionally include one or more temperature sensor(s). As shown in FIG. 10, the energy attenuating device 50 may include one or more temperature sensors 102, such as thermistor(s) or thermocouple(s), attached to or embedded within the energy attenuating device 50 that monitor the temperature of the energy attenuating device 50. A non-transitory medium 90 connected to the temperature sensors 102 and accessible by the processing unit 100, may be used to store one or more temperatures recorded by the temperature sensors 102. Such temperature data can be used to compensate for varying material properties of the energy attenuating device 50 as to provide a more accurate measurement of power by the testing apparatus 10. For instance, the attenuation of some materials comprising the energy attenuating device 50 can increase with increasing temperature, such that the energy attenuating device 50 may attenuate more energy at higher temperatures than it would at lower temperatures. Therefore, such effects in the power measurement may be accounted for by using the temperature sensors 102 to determine the temperature of the energy attenuating device 50 prior to the delivery of ultrasound energy by the ultrasound device 101. Additionally, because the attenuation of ultrasonic energy will increase the temperature of the energy attenuating device 50, temperature measurements recorded by the temperature sensor(s) 102 can provide an indirect measure of the power delivered by the ultrasound device 101. In one embodiment, the processing unit 100 is configured to determine the temperature change of the energy attenuating device 50 by subtracting the temperatures recorded by the temperature sensors 102 before and after the ultrasound delivery to determine the power delivered by the ultrasound device 101.

In the above embodiments, the sensor(s) is described as being configured to sense one or more temperatures at the energy-attenuating device 50. In other embodiments, the sensor(s) may be configured to sense other characteristic(s) at the energy-attenuating device 50. Accordingly, the processing unit may be configured to obtain a first value from a sensor (e.g., before a delivery of energy towards the energy-attenuating device 50), obtain a second value from the sensor (e.g., after the delivery of energy towards the energy-attenuating device 50), and determine a difference between the first value and the second value, wherein the first value may represent a temperature, or another characteristic, at the energy-attenuating device 50.

Processing System Architecture

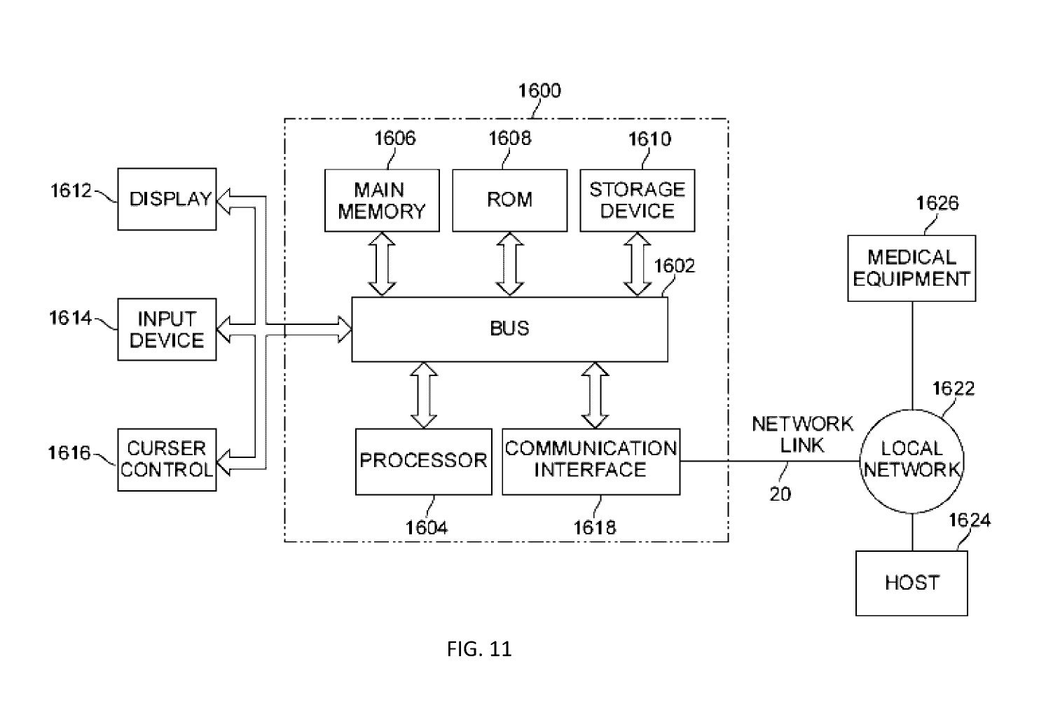

FIG. 11 is a block diagram illustrating an embodiment of a specialized processing system 1600 that can be used to implement various embodiments described herein. For example, the processing system 1600 may be configured to implement the method of FIG. 6, FIG. 7, FIG. 8, or any combination of the foregoing, in accordance with some embodiments. Also, in some embodiments, the processing system 1600 may be used to implement the processing unit in the controller 14 of FIG. 1. For example, the processing unit that is a part of the controller 14 controlling the ultrasound transducer 12 may be implemented using the processing system 1600.

Referring to FIG. 11, the processing system 1600 includes a bus 1602 or other communication mechanism for communicating information, and a processor 1604 coupled with the bus 1602 for processing information. The processor 1604 may be an example of the processing unit 100 of FIG. 5 or an example of any processor described herein. The processing system 1600 also includes a main memory 1606, such as a random access memory (RAM) or other dynamic storage device, coupled to the bus 1602 for storing information and instructions to be executed by the processor 1604. The main memory 1606 also may be used for storing temporary variables or other intermediate information during execution of instructions to be executed by the processor 1604. The processing system 1600 further includes a read only memory (ROM) 1608 or other static storage device coupled to the bus 1602 for storing static information and instructions for the processor 1604. A data storage device 1610, such as a magnetic disk or optical disk, is provided and coupled to the bus 1602 for storing information and instructions.

The processing system 1600 may be coupled via the bus 1602 to a display 1612, such as a cathode ray tube (CRT), for displaying information to a user. An input device 1614, including alphanumeric and other keys, is coupled to the bus 1602 for communicating information and command selections to processor 1604. Another type of user input device is cursor control 1616, such as a mouse, a trackball, or cursor direction keys for communicating direction information and command selections to processor 1604 and for controlling cursor movement on display 167. This input device typically has two degrees of freedom in two axes, a first axis (e.g., x) and a second axis (e.g., y), that allows the device to specify positions in a plane.

In some embodiments, the processing system 1600 can be used to perform various functions described herein. According to some embodiments, such use is provided by processing system 1600 in response to processor 1604 executing one or more sequences of one or more instructions contained in the main memory 1606. Those skilled in the art will know how to prepare such instructions based on the functions and methods described herein. Such instructions may be read into the main memory 1606 from another processor-readable medium, such as storage device 1610. Execution of the sequences of instructions contained in the main memory 1606 causes the processor 1604 to perform the process steps described herein. One or more processors in a multi-processing arrangement may also be employed to execute the sequences of instructions contained in the main memory 1606. In alternative embodiments, hard-wired circuitry may be used in place of or in combination with software instructions to implement the various embodiments described herein. Thus, embodiments are not limited to any specific combination of hardware circuitry and software.

The term “processor-readable medium” as used herein refers to any medium that participates in providing instructions to the processor 1604 for execution. Such a medium may take many forms, including but not limited to, non-volatile media, volatile media, and transmission media. Non-volatile media includes, for example, optical or magnetic disks, such as the storage device 1610. A non-volatile medium may be considered an example of non-transitory medium. Volatile media includes dynamic memory, such as the main memory 1606. A volatile medium may be considered an example of non-transitory medium. Transmission media includes coaxial cables, copper wire and fiber optics, including the wires that comprise the bus 1602. Transmission media can also take the form of acoustic or light waves, such as those generated during radio wave and infrared data communications.

Common forms of processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a processor can read.

Various forms of processor-readable media may be involved in carrying one or more sequences of one or more instructions to the processor 1604 for execution. For example, the instructions may initially be carried on a magnetic disk of a remote computer. The remote computer can load the instructions into its dynamic memory and send the instructions over a telephone line using a modem. A modem local to the processing system 1600 can receive the data on the telephone line and use an infrared transmitter to convert the data to an infrared signal. An infrared detector coupled to the bus 1602 can receive the data carried in the infrared signal and place the data on the bus 1602. The bus 1602 carries the data to the main memory 1606, from which the processor 1604 retrieves and executes the instructions. The instructions received by the main memory 1606 may optionally be stored on the storage device 1610 either before or after execution by the processor 1604.

The processing system 1600 also includes a communication interface 1618 coupled to the bus 1602. The communication interface 1618 provides a two-way data communication coupling to a network link 1620 that is connected to a local network 1622. For example, the communication interface 1618 may be an integrated services digital network (ISDN) card or a modem to provide a data communication connection to a corresponding type of telephone line. As another example, the communication interface 1618 may be a local area network (LAN) card to provide a data communication connection to a compatible LAN. Wireless links may also be implemented. In any such implementation, the communication interface 1618 sends and receives electrical, electromagnetic or optical signals that carry data streams representing various types of information.

The network link 1620 typically provides data communication through one or more networks to other devices. For example, the network link 1620 may provide a connection through local network 1622 to a host computer 1624 or to equipment 1626 such as a radiation beam source or a switch operatively coupled to a radiation beam source. The data streams transported over the network link 1620 can comprise electrical, electromagnetic or optical signals. The signals through the various networks and the signals on the network link 1620 and through the communication interface 1618, which carry data to and from the processing system 1600, are exemplary forms of carrier waves transporting the information. The processing system 1600 can send messages and receive data, including program code, through the network(s), the network link 1620, and the communication interface 1618.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

1. An apparatus for testing an ultrasound device having an ultrasound transducer and a controller, the apparatus comprising:
a housing;
an absorbing layer inside the housing, wherein the absorbing layer is configured to receive ultrasound energy from the ultrasound transducer; and
a thermal camera for detecting temperature at the absorbing layer.

2. The apparatus of claim 1, wherein the ultrasound transducer is configured to deliver the ultrasound energy sequentially to a plurality of target areas at the absorbing layer.

3. The apparatus of claim 2, wherein the target areas are arranged in a circular pattern, an elliptical pattern, a linear pattern, or any other defined pattern.

4. The apparatus of claim 1, wherein the absorbing layer comprises a material that can withstand temperature ranging from 0° C. to 300° C.

5. The apparatus of claim 1, wherein the housing comprises a compartment containing fluid, and the absorbing layer comprises a material with an acoustic velocity that is less than an acoustic velocity of the fluid.

6. The apparatus of claim 1, wherein the absorbing layer comprises a Teflon material.

7. The apparatus of claim 1, wherein the absorbing layer comprises a material that includes urethane, silicone, graphite, plastic, or any combination of the foregoing.

8. The apparatus of claim 7, wherein the material is mixed with one or more fillers selected from the group consisting of polymeric microspheres, glass microspheres, boron-nitride, oxides, and graphite.

9. The apparatus of claim 1, further comprising an energy-attenuating device positioned between the ultrasound transducer and the absorbing layer, the energy-attenuating device configured to attenuate ultrasound energy provided by the ultrasound transducer to reduce an amount of the ultrasound energy received by the absorbing layer

10. The apparatus of claim 9, wherein the energy-attenuating device comprises two or more layers.

11. The apparatus of claim 10, wherein the two or more layers comprise a first layer having a first thickness and a second layer having a second thickness, the first thickness being different from the second thickness.

12. The apparatus of claim 9, wherein the energy-attenuating device comprises a silicone based product mixed with boron-nitride.

13. The apparatus of claim 9, wherein the energy-attenuating device comprises a plastic, urethane, or silicone material that is mixed with one or more fillers selected from the group consisting of polymeric microspheres, glass microspheres, boron-nitride, oxide, and graphite.

14. The apparatus of claim 9, wherein the energy-attenuating device comprises a natural product.

15. The apparatus of claim 9, further comprising one or more sensors attached to the energy-attenuating device.

16. The apparatus of claim 15, wherein the one or more sensors are configured to sense one or more temperatures at the energy-attenuating device.

17. The apparatus of claim 15, further comprising a processing unit configured to obtain a first value from one of the one or more sensors, obtain a second value from the one of the one or more sensors, and determine a difference between the first value and the second value.

18. The apparatus of claim 1, further comprising a fiducial marker that is detectable using ultrasound imaging.

19. The apparatus of claim 18, wherein the fiducial marker comprises a metal or plastic object attached to the absorbing layer or to a component located in the housing.

20. The apparatus of claim 18, wherein the ultrasound device is configured to determine a position of the fiducial marker, and to operate the ultrasound transducer based on the determined position.

21. The apparatus of claim 1, further comprising a processing unit configured to analyze thermal image data from the thermal camera to perform a power test for the ultrasound device.

22. The apparatus of claim 21, wherein the processing unit is configured to calculate a first mean temperature for a first region-of-interest.

23. The apparatus of claim 22, wherein the processing unit is configured to calculate a second mean temperature for a second region-of-interest.

24. The apparatus of claim 21, wherein the processing unit is configured to determine a first set of data representing how temperature varies through time for a first region-of-interest.

25. The apparatus of claim 24, wherein the processing unit is configured to determine a second set of data representing how temperature varies through time for a second region-of-interest.

26. The apparatus of claim 21, wherein the processing unit is configured to determine a maximum temperature, a mean temperature, a slope of a temperature-vs-time curve, an integral value of temperatures through space, an integral value of temperatures through time, or any combination of two or more of the foregoing.

27. The apparatus of claim 1, further comprising a processing unit configured to analyze thermal image data from the thermal camera to perform a targeting test for the ultrasound device.

28. The apparatus of claim 27, wherein the thermal image data is resulted from the ultrasound transducer delivering the ultrasound energy to multiple target areas in a defined pattern, and wherein the processing unit is configured to:
determine locations of the respective target areas based on the thermal image data;
calculate a mean location of the locations of the respective target areas; and
determine a difference between the mean location and a target location to obtain an overall targeting error for the defined pattern.

29. The apparatus of claim 28, wherein the processing unit is also configured to:
determine a difference between the mean location and the location of at least one of the target areas to obtain a targeting error for the at least one of the target areas.

30. The apparatus of claim 28, wherein the target areas are arranged in a circular pattern, an elliptical pattern, a linear pattern, or any other defined pattern.

31. The apparatus of claim 1, further comprising a processing unit configured to:
obtain a first baseline temperature for a first region-of-interest before the ultrasound transducer is operated to deliver energy aiming at a first target area;
obtain a first temperature data for the first region-of-interest after the ultrasound transducer is operated to deliver energy aiming at the first target area; and
determine a first difference between the first temperature data and the first baseline temperature to obtain a first delta temperature.

32. The apparatus of claim 31, wherein the processing unit is further configured to:
obtain a second baseline temperature for a second region-of-interest before the ultrasound transducer is operated to deliver energy aiming at a second target area;
obtain a second temperature data for the second region-of-interest after the ultrasound transducer is operated to deliver energy aiming at the second target area; and
determine a second difference between the second temperature data and the second baseline temperature to obtain a second delta temperature.

33. The apparatus of claim 1, further comprising a processing unit configured to analyze thermal image data from the thermal camera to perform a power test and a targeting test for the ultrasound device.

34. The apparatus of claim 1, further comprising a non-transitory medium for storing one or more images from the thermal camera.

35. The apparatus of claim 1, further comprising a non-transitory medium for storing temperature data obtained using the thermal camera.

36. The apparatus of claim 1, further comprising a processing unit for determining power performance of the ultrasound device based on output from the thermal camera.

37. The apparatus of claim 1, further comprising a processing unit for determining targeting performance of the ultrasound device based on output from the thermal camera.

38. The apparatus of claim 1, further comprising a processing unit for determining power performance and targeting performance of the ultrasound device based on output from the thermal camera.

39. The apparatus of claim 1, further comprising a camera holder for holding the thermal camera.

40. The apparatus of claim 39, further comprising a mounting component at or coupled to the housing for allowing the camera holder to be detachably secured thereto.

41. The apparatus of claim 39, wherein the mounting component comprises a tubular structure defining a space for accommodating at least a part of the camera holder.

42. The apparatus of claim 40, wherein the housing comprises a cover, and the mounting component is located at the cover.

43. The apparatus of claim 1, wherein the housing defines a space for holding fluid.

44. The apparatus of claim 1, wherein the housing includes side walls defining a perimeter of the housing, and a lid for covering an end of the housing.

45. The apparatus of claim 1, wherein the housing comprises a mounting bracket.

46. The apparatus of claim 45, wherein the mounting bracket is configured to align with the ultrasound transducer and to secure the apparatus to the ultrasound transducer.

47. A method for testing an ultrasound device having an ultrasound transducer and a controller, comprising:
operating the ultrasound transducer to deliver ultrasound energy towards an absorbing layer at a testing apparatus;
using a thermal camera to detect temperature at the absorbing layer;
obtaining thermal image data from the camera; and
analyzing the thermal image data to determine whether the ultrasound device is operating desirably.

48. The method of claim 47, further comprising using an energy-attenuating device positioned between the ultrasound transducer and the absorbing layer to attenuate the ultrasound energy delivered by the ultrasound transducer to reduce the ultrasound energy incident at the absorbing layer.

49. The method of claim 48, wherein the energy-attenuating device comprises two or more layers.

50. The method of claim 49, wherein the two or more layers comprise a first layer having a first thickness and a second layer having a second thickness, the first thickness being different from the second thickness.

51. The method of claim 48, wherein the energy-attenuating device comprises at least four layers.

52. The method of claim 48, wherein the energy-attenuating device comprises a silicone based product mixed with boron-nitride.

53. The method of claim 48, wherein the energy-attenuating device comprises a plastic, urethane, or silicone material that is mixed with one or more fillers selected from the group consisting of polymeric microspheres, glass microspheres, boron-nitride, oxide, and graphite.

54. The method of claim 48, wherein the energy-attenuating device comprises a natural product.

55. The method of claim 47, wherein the ultrasound transducer is operated to deliver the ultrasound energy sequentially to a plurality of target areas at the absorbing layer.

56. The method of claim 55, wherein the target areas are arranged in a circular pattern, an elliptical pattern, a linear pattern, or any other defined pattern.

57. The method of claim 47, wherein the absorbing layer comprises a material that can withstand temperature ranging from 0° C. to 300° C.

58. The method of claim 47, further comprising providing a container of fluid for coupling the ultrasound energy to the absorbing layer, wherein the absorbing layer comprises a material with an acoustic velocity that is less than the velocity of the fluid.

59. The method of claim 47, wherein the absorbing layer comprises a Teflon material.

60. The method of claim 47, wherein the absorbing layer comprises a material that includes urethane, silicone, graphite, plastic, or any combination of the foregoing.

61. The method of claim 60, wherein the material of the absorbing layer is mixed with one or more fillers selected from the group consisting of polymeric microspheres, glass microspheres, boron-nitride, oxide, and graphite.

62. The method of claim 47, wherein the act of analyzing is for performing a power test for the ultrasound device.

63. The method of claim 47, wherein the act of analyzing comprises calculating a first mean temperature for a first region-of-interest.

64. The method of claim 63, wherein the act of analyzing further comprises calculating a second mean temperature for a second region-of-interest.

65. The method of claim 47, wherein the act of analyzing is performed to determine a first set of data representing how temperature varies through time for a first region-of-interest.

66. The method of claim 65, wherein the act of analyzing is performed to determine a second set of data representing how temperature varies through time for a second region-of-interest.

67. The method of claim 47, wherein the act of analyzing comprises determining a maximum temperature, a mean temperature, a slope of a temperature-vs-time curve, an integral value of temperatures through space, an integral value of temperatures through time, or any combination of two or more of the foregoing.

68. The method of claim 47, wherein the act of analyzing is for performing a targeting test for the ultrasound device.

69. The method of claim 47, wherein the ultrasound transducer is operated to deliver the ultrasound energy to multiple target areas in a defined pattern, and wherein the act of analyzing comprises:
determining locations of the respective target areas based on the thermal image data;
calculating a mean location of the locations of the respective target areas; and
determining a difference between the mean location and a target location to obtain an overall targeting error for the defined pattern.

70. The method of claim 69, further comprising determining a difference between the mean location and the location of at least one of the target areas to obtain a targeting error for the at least one of the target areas.

71. The method of claim 47, wherein the target areas are arranged in a circular pattern, an elliptical pattern, a linear pattern, or any other defined pattern.

72. The method of claim 47, wherein the ultrasound transducer is operated to deliver energy aiming at a first target area and a second target area at the absorbing material, and wherein the act of analyzing comprises:
obtaining a first baseline temperature for a first region-of-interest before the ultrasound transducer is operated to deliver energy aiming at the first target area;
obtaining a first temperature data for the first region-of-interest after the ultrasound transducer is operated to deliver energy aiming at the first target area; and
determining a first difference between the first temperature data and the first baseline temperature to obtain a first delta temperature.

73. The method of claim 72, wherein the act of analyzing further comprises:
obtaining a second baseline temperature for a second region-of-interest before the ultrasound transducer is operated to deliver energy aiming at the second target area;
obtaining a second temperature data for the second region-of-interest after the ultrasound transducer is operated to deliver energy aiming at the second target area; and
determining a second difference between the second temperature data and the second baseline temperature to obtain a second delta temperature.

74. The method of claim 47, wherein the thermal image data is analyzed to perform a power test and a targeting test for the ultrasound device.

75. The method of claim 47, further comprising providing a tank of fluid between the ultrasound transducer and the absorbing layer.
